(19) United States
(12) Patent Application Publication
(10) Pub. No.: US 2002/0164652 A1
(43) Pub. Date: Nov. 7, 2002

(54) Conformational antigens and antibodies recognizing said antigens, a process for the efficient generation of monoclonal antibodies to native or conformational antigens expressed or carried by eukaryotic cells, a process for the selection of conformational antigens, use of monoclonal antibodies for therapeutical, diagnostic or vaccine applications

(76) Inventors: Jurg Gysin, St. Zacharie (FR); Artur Scherf, Paris (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(21) Appl. No.: 10/082,051

(22) Filed: Feb. 25, 2002

Related U.S. Application Data
(60) Provisional application No. 60/270,581, filed on Feb. 23, 2001.

(51) Int. Cl. G01N 33/53; C12P 21/04; C07K 16/44

(52) U.S. Cl. 435/7.1; 435/70.21; 530/388.15

(57) ABSTRACT

Conformational antigens and antibodies recognizing said antigens, a process for the efficient generation of monoclonal antibodies to native or conformational antigens expressed or carried by eukaryotic cells. A process for the selection of conformational antigens, use of monoclonal antibodies for therapeutical, diagnostic or vaccine applications.

The process for preparing monoclonal antibodies, comprises:

rendering an animal tolerant to an eukaryotic cell in a first state;

detecting said tolerant animal;

immunizing said tolerant animal, by injection of the eukaryotic cell in a second state carrying a neo-antigen or a non-self antigen;

fusing B cells of said immunized mice with a myeloma cell line; and

selecting the hybridoma expressing antibodies against said neo-antigen or non-self antigen.

### CONTINUING APPLICATION DATA

This application claims benefit to U.S. provisional application serial No. 60/270,581, filed on Feb. 23, 2001, and incorporated herein by reference.

### BACKGROUND OF THE INVENTION

1. Field of the Invention

The present invention is directed to a process for preparing monoclonal antibodies and to utilization of the monoclonal antibodies prepared by this process for purpose of diagnostic, therapy including vaccine purpose, identification of native or conformational antigens, and the use thereof for the induction of an immune response. More particularly, the present invention relates to a process for preparing monoclonal antibodies raised against specific antigens expressed or carried by eukaryotic cells, as native or conformational antigens, minor antigens and poorly immunogenic antigens, and utilization of the monoclonal antibodies, for example, for the selection of conformational antigens, immunization, therapy, and diagnostic purposes.

2. Background of the Invention

The systematic identification of native or conformational, or minor or poorly immunogenic surface antigens is a serious technical problem. Except by using crystallography techniques, it is very difficult to locate the different residues of an antigen involved in the conformation of a molecule of diagnostic or therapeutic, vaccine, interest.

Many patents and scientific articles describe the preparation and selection of poly or monoclonal antibodies specific for an antigen but the antigen is very rarely a conformational antigen because the technology used prior to the present invention does not permit reproducible selection of conformational or native antigen.

The development of monoclonal antibodies (mabs) directed against native and conformational surface molecules such as tumor markers on cancer cells or pathogen derived surface antigen on infected host cells is hampered by the abundance of host cell surface molecules. Similarly immuno-dominant antigens induce an overwhelming antibody response and make it extremely difficult to develop monoclonal antibodies (mabs) against minor antigens or antigens that are poorly immunogenic. This is the case in a number of human and animal pathogens that infect different kind of host cells such as a number of viruses (such as hepatitis virus, rabies virus or HIV) or intracellular protozoan parasites (such as the human Plasmodium species, Babesia species which infect cattle/dog erythrocytes or Leishmania).

As a general application, monoclonal antibodies are useful for the detection of antigens associated with particular pathological events, e.g. diagnostic markers of cancer and adhesion molecule markers in certain pathological cases of malaria etc. It is also possible to humanize mabs for clinical therapeutic use (Ren, 1991). For instance the infected/modified cell carrying this marker can be labeled in vivo. This approach is of potential use for treating people suffering, for example, from a parasitic or viral infection.

Robert et al. (1995) have described a process to obtain antibodies against a surface receptor for P. falciparum of Saimiri brain endothelial cells (SBEC). The central event in the pathogenesis of severe P. falciparum infection is the sequestration of P. falciparum parasitized erythrocytes in the microvasculature of different organs. The process is mediated by specific adherence ligands present on the infected erythrocytes surface and different host receptors expressed on the membrane of microvascular endothelial cells.

To obtain antibodies against the host receptors involved in the adhesion process of P. falciparum expressed on the membrane of microvascular endothelial cells, Robert et al. have previously rendered mice tolerant to P. falciparum infected red blood cells (PRBC). Then, they have immunized these PRBC tolerant mice with a complex of PRBC/SBEC after mechanical or chemical disruption of SBEC, in order to immunize the tolerant mice against a SBEC P. falciparum receptor.

A disadvantage of this process is that the polyclonal or monoclonal antibodies generated are directed against endothelial cells of the host and exclude therapeutic use which would lead to the destruction of the host cells.

Another disadvantage of this process is that the antibodies are not generated against conformational or native antigens due to the dissociation of PRBC from SBEC.

The problem of raising specific monoclonal antibodies (mabs) against nature or conformational antigens expressed or present at the surface of a cell constitute one of the problem solved by the instant invention.

Despite intensive research in many different laboratories in the world for the past 15 years, only very few attempts to raise mabs against conformational antigens expressed or carried at the surface have been successful. In fact only one paper related to a conformational antigen of P. falciparum infected erythrocytes is known by the inventors and the process for the selection of conformational antigen is very different from the process according to the invention described herein (Smith et al. 1995).

Another problem solved by the present invention concerns raising specific monoclonal antibodies to minor or poorly immunogenic antigens.

### SUMMARY OF THE INVENTION

A possibility to circumvent this problem is to render animals, e.g. mice, immuno-tolerant (based on the absence of a humoral response) against surface antigens by injecting in newborn animals e.g. Balb/c mice (24 to 48 hours of age) an overwhelming quantity of antigens expressed by the cell of interest in a first state.

For example it is possible to render mice tolerant for human erythrocytes or CHO cells. This treatment will induce an immunological tolerance rendering later the animal incapable to build up an efficient B cell response against the same set of cell surface antigens.

The use of such B cell tolerant mice for the immunization against one or several different antigens is particularly appropriate for the development of antibodies directed against native, minor, poorly immunogenic or conformational epitopes. This immunization concept is applicable for any cell surface modification induced by pathogens such as fungi, parasites, virus, bacteria, etc. or by a normal or pathological development of the cell.

For example, when immunizing such normal human O⁺ erythrocytes tolerant mice with, e.g., P. falciparum parasitized human O⁺ erythrocytes, these mice will almost exclusively build up an antibody mediated immune response against additional antigens due to the P. falciparum infection, e.g. PfEMP-1/var, not present on normal human O₊ erythrocytes. This antibody can be of various isotypes.

The antibody selected antigens according to the process of the present invention can be used for the design of new therapeutical molecules.

For example, antibodies to the surface antigens of P. falciparum infected erythrocytes can inhibit their adhesion to host endothelial cells, an event involved in pathology of malaria. A frequently used technique is to express a molecule of interest on the surface of CHO cells. In many instances, CHO cells express recombinant molecules in a functional or antigenic form that resembles the one described for the native molecule. Examples can be found in European patent EP 0 356 109 for HIV virus and in U.S. Pat. Nos. 5,326,513 and 6,051,426 for hepatitis virus. The procedure of developing mabs to the recombinant surface molecule according to the present invention is the same as for infected erythrocytes.

Importantly, mabs raised against the antigens of interest present on the cell surface can be used as a screening procedure for the detection and identification of new surface antigens, especially native, conformational, minor or poorly immunogenic antigens.

One object of the present invention is a method for eliciting monoclonal antibodies recognizing native or conformational structures, such as a peptide, or lipopeptidic, or glycoprotein or sugar moieties, as antigen.

Another object of the present invention is a method for the selection and purification of conformational or native structures by using said monoclonal antibodies. Random peptide libraries can be used for the selection of ligands reacting specifically with the monoclonal antibodies of the invention. As example, a method for the preparation of a random library is disclosed in Felici et al., J. Mol. Biol. 1991, 222.

Another object of the invention is the use of the conformational structures for diagnostic, vaccine, or therapeutic purpose as will as, for the selection of drugs interacting with said conformational or native structures or for the design of new therapeutic molecules.

Another object of the invention is the use of the monoclonal antibodies elicited by the method of the invention for diagnostic or therapeutic including vaccine, purposes.

Another object of the invention is the use of the monoclonal antibodies for the targeting of eukaryotic cells carrying a neo-antigen or a non-self antigen. The target can also involve a toxic molecule coupled to the monoclonal antibody in order to destroy selectivity the targeted cells in vitro, in vivo, or ex vivo.

An object of the invention is a process of treatment of a fluid of a patient by (i) contacting said fluid with monoclonal antibodies recognizing the non-self or neo-antigen at the surface of the eukaryotic cells of the patient, and (ii) separating the formed complex by the monoclonal antibodies and said antigen from the patient's fluid.

### DETAILED DESCRIPTION OF THE INVENTION

By native antigens it is intended in the present application surface antigens as they occur in the normal sate of the cell, i.e., antigens that maintain their biological function and conformation in physiological conditions.

By conformational antigens it is intended in the present application surface antigens as they occur in the normal state of the cell, i.e. antigens that maintain their biological function in physiological conditions.

By neo-antigen or non-self antigen, it is intended in the present application, an antigen that was not present on the surface of the cell in a first state that is at a given moment of the differentiation state or life cycle state at time t and which arises at the surface of the cell in a second state, that is at a differentiation or life cycle state different from the one of the first state at t+1; said antigens represent a specific state of the cell:

normal cell versus cancer cell,

non-infected cell versus infected cell,

immature cell versus differentiated cell.

Accordingly, the present invention provides process for preparing monoclonal antibodies, comprising:

rendering an animal tolerant to an eukaryotic cell in a first state;

detecting said tolerant animal;

immunizing said tolerant animal, by injection of the eukaryotic cell in a second state carrying a neo-antigen or a non-self antigen;

fusing B cells of said immunized mice with a myeloma cell line; and

selecting the hybridoma expressing antibodies against said neo-antigen or non-self antigen.

Rendering an animal, for instance a mouse tolerant (step (a)) to an eukaryotic cell can be performed by any known process. This can be performed for example by sub-cutaneous injections of an appropriate preparation of eukaryotic cells of interest. Advantageously, a first injection is performed on new-born mice, followed by a second injection (the boost injection) several weeks after the first one. The second injection can take place between 2 to 4 weeks after the first one, advantageously 3 weeks after the first one.

The quantity of cells which are injected in the first step can vary due to the cell itself. One skilled in the art knows how to adjust the exact quantity necessary to obtain the best result. For example, for CHO cells or for erythrocytes, the quantity can vary from 10⁵ to 10¹⁰ cells, advantageously from 10⁶ to 10⁹ cells.

Detecting tolerant mouse (step (b)) can be performed by any known process. For example it is possible to verify the absence of antibodies against the surface of the cells in a first state in the serum of the animals.

Immunizing said tolerant mice (step (c)) by the eukaryotic cell (cell of interest) carrying a neo-antigen or a non-self antigen can be performed by any known process.

This step (c) can be performed for example by injections (sub-cutaneous, intramuscular, intra-venous) of an appropriate preparation of eukaryotic cells of interest. Advantageously, a first injection is performed on mice, followed by a second injection several weeks after the first one. The first immunization injection can take place between 5 to 7 weeks, advantageously 6 weeks, after the first injection of step (a). The second immunization injection of step c can take place between 8 to 10 weeks, advantageously 9 weeks, after the first injection of step (a). A third immunization injection can eventually be performed.

Fusing B cells (step (d)) of said immunized mice with a myeloma cell line and selecting the hybridoma expressing antibodies against said neo-antigen or non-self antigen can be performed by any known process. For example the techniques described by Galfre et al. (1981) or Kohler G et al. (1975) can be applied.

In another embodiment the process described above also includes:

(f) optionally culturing the selected hybridoma and purifying the monoclonal antibodies.

In another embodiment of the invention, the antibodies are further humanized. Humanization can be performed as described by Emery, 1995.

The most effective method of archiving humanization is a reshaping technology of Winter and Colleagues (Verhoeyen, M., Milstein, C. and Winter, G. 1988 Science 239: 1098-1104) using the detailed methods described recently (Gussow, D, and Seeman, G., 1991, Method. Enzymol. 203: 99-121). The steps involved, starting with a murine hybridoma cell line are as followed: 1. Cloning the immunoglobulin variable region segments. 2. Identification of hypervariable loop regions. 3. ‘CDR’ grafting of the mouse antibody gene in the gene of the human variable-chain acceptor framework. 4. Assembly into a mammalian expression vector and expression of humanized antibody in a mammalian cell line. Antibody is synthesized and secreted from such cells.

The present invention also provides monoclonal antibody susceptible to be prepared by the process described above.

The present invention also provides an antigen especially a native or a conformational antigen, capable of reacting with a monoclonal antibody prepared by the process described above.

The present invention also provides a process for screening an active molecule capable of reacting specifically with the monoclonal antibody described above. The present invention also provides a process for selecting a native or conformational antigen, comprising:

a) rendering an animal tolerant to an eukaryotic cell in a first state;

b) detecting said tolerant animal;

c) immunizing said tolerant animal, by injecting said eukaryotic cell in a second state carrying a neo-antigen or a non-self antigen;

d) preparing an hybridoma against said neo-antigen or non-self antigen;

e) selecting the hybridoma expressing antibodies against said neo-antigen or non-self antigen;

f) contacting the monoclonal antibody produced by the hybridoma of (e) with an antigenic preparation; and

g) selecting the complex formed between said monoclonal antibody and the conformational native antigen of interests.

In another embodiment the process described above also includes:

revealing the complex.

optionally, separating the antibody from the antigen from the complex.

Steps (a)-(e) of the said process can be performed as previously described. Other steps can be performed as described in the literature.

In a preferred embodiment, the animal is a murine animal; in a most preferred embodiment, the animal is a mouse.

In another embodiment of the invention, the neo-antigen or non-self antigen is selected from the group consisting of bacteria, fungi, parasitic and cancer antigens and antigens induced by the normal or pathological development of the cell.

In another embodiment of the invention the active molecule described above is a component for a diagnostic detection of the presence or absence of antibodies in a serum of an animal including a human.

In another embodiment of the invention, the active molecule described above can compete with the neo or non-self antigen of the virus, the bacteria, the fungi, the parasite or the cancer present at the surface of cells or induced by the normal or pathological development of the cell.

In another embodiment of the invention, the active molecule described above is capable of inducing an immune response in vivo or in vitro against a bacterial or viral, fungal or parasite infection against a cancer or any pathological development of the cell inducing neo-antigen development.

The present invention also concerns the use of the human or the animal antibody described above in the preparation of a composition for the immunization or the treatment of a human or an animal for a virus, bacteria, fungi or parasite infection or cancer.

The present invention also concerns the use of the human or the animal antibody described above in the preparation of a composition for diagnosing a viral, bacterial, parasite or fungal infection, a cancer or any development of the cell inducing neo-antigen development.

The present invention also provides a process for targeting eukaryotic cells carrying a neo-antigen or a non-self antigen wherein said process uses monoclonal antibodies directed against said neo-antigen or non-self antigen obtained by the process described above.

In a specific embodiment of the invention, the monoclonal antibodies are further labeled.

In another specific embodiment of the invention, the monoclonal antibodies are further coupled to a molecule toxic for the targeted cells. The invention also provides hybridoma according to step (e), expressing antibodies against neo-antigens or non-self antigens. The present invention also provides the hybridoma Pf 26G1/B4 deposited at Collection Nationale de Cultures de Microorganismes (CNCM) on Feb. 23, 2001, under accession number I-2635.

The present invention also provides the hybridoma Pf 26G1/C 10 deposited at Collection Nationale de Cultures de Microorganismes (CNCM) on Feb. 23, 2001, under accession number I-2636.

In a specific embodiment of the invention, the frequency of obtained hybridoma cell lines having the property of recognizing selectively a conformational antigen or a native antigen is up to 200 times greater than hybridoma cell lines obtained by classical techniques.

The present invention also provides process for screening active molecule capable of reacting specifically with the conformational, native, minor or poorly immunogenic antigen obtained by the process described above.

The present invention also provides a hybridoma which secretes an antibody having the same epitope specificity as the antibody produced by hybridoma Pf 26G1/B4 deposited at Collection Nationale de Cultures de Microorganismes (CNCM) on Feb. 23, 2001, under accession number 1-2635.

The present invention also provides a hybridoma which secretes an antibody having the same epitope specificity as the antibody produced by hybridoma Pf 26G1/C10 deposited at Collection Nationale de Cultures de Microorganismes (CNCM) on Feb. 23, 2001, under accession number I-2636.

The technique developed for screening of antibodies directed against surface antigens is for example the commonly used cell surface immunofluorescence assay (IFA) (liquid phase IFA at 4 C). Secondary fluorescent antibodies are absorbed against the uninfected host cell in order to increase the specificity of the detection system.

The score of mice which became tolerant after the injection of host cells is variable. Approximately 10 to 40% of them did not develop antibodies judged by liquid phase IFA (at 4 C for a plasma dilution of ¼). Another 20 to 40% developed a faint immunofluorescence (IF) pattern concerning cellular surface antigens and the other mice present different types of IF intensities. The best results for a specific immunization with infected host cells (P. falciparum infected erythrocytes and transfected CHO cells expressing a P. falciparum surface adhesion molecule) has been obtained with IF “negative” animals but relatively satisfying results can also be achieved with animals that developed a faint IF positive response.

The relative score of specific mabs against conformational antigens present on the cell surface is generally high. Typically, between 10 to 30 IFA positive wells for a total of 50 wells screened (after fusion) were observed in the case of mabs developed against P. falciparum surface antigens of infected red blood cells (RBCs). A genetic restriction to respond against an antigen could be solved by using different mice lines.

The invention also relates to a conformational antigen selected and characterized by its capacity to react with monoclonal antibody obtained by a process which is 200 uptimes greater successful than a classical process to obtain similar hybridoma.

The invention also relates to a kit of detection of antigens, comprising at least a monoclonal antibody obtained by the process of preparation of monoclonal antibodies as described here above.

### EXAMPLES

Having generally described this invention, a further understanding can be obtained by reference to certain specific examples which are provided herein for purposes of illustration only and are not intended to be limiting unless otherwise specified.

### Example 1:

Materials and Methods

Parasites

P. falciparum strains B358, BXII, FCBP, Suk, H, IBR, FCR3, were cultured and maintained under standard culture conditions as previously described by Pouvelle et al., (1998) replacing 10% human serum with 5% Albumax.

Tissue cryosections of 6 P. falciparum-infected placentas from Cameroonian women (n° 24, 42, 42DJ, 193, 939 and 940) have been described by Gysin J. et al. (1999).

CHO-Transfectant

CHO-745 cells and a transfectant of this cell line expressing the DBL-γ3 domain of var^{CSA} at its surface were obtained and maintained as previously described in WO 00.116326.

Placenta Cryosections

Fresh malaria placenta biopsy samples about 5×5×5 mm in size were obtained from the same 6 Cameroonian women from whom the parasite populations listed above were obtained by flushing with CSA (Gysin J. et al. (1999)). They were snap frozen immediately after delivery and stored in liquid nitrogen until use. For 1-IFA, we used 7 μm unfixed placenta cryosections mounted on standard microscope slides.

Selection of CSA, CD36 and ICAM- 1 Adhesive Phenotype

Highly synchronized (4+2 hours) parasites in mature blood-stage-infected erythrocytes of the CSA adhesive phenotype (mIE^{CSA}) were obtained by regular panning on Sc17 Saimiri brain microvascular endothelial cells as described by Gay F et al. (1995), and successive sorbitol treatments (Lambros C. et al. 1979).

The adhesive specificity of such mIE^{CSA} was investigated by using concentrated synchronized parasites obtained by gelatin flotation using Plasmagel (Heidrich HG. et al. (1982)). These parasites were incubated with a CSA chain bearing recombinant human thrombomodulin-coated magnetic beads (Dynabeads M450, Dynal ASA, Oslo, Norway), as described elsewhere (Parzy D. et al. (2000); Fusai T. et al. (2000)). Bound mIE were expanded in culture and cytoadhesion inhibition assays were regularly performed (Robert C. et al. (1995)) to assess the specificity of binding to CSA. Typically, the adhesion of mIE selected in this way was inhibited by more than 95 %, by 100 μg/ml of soluble CSA (Fluka, l'Isle Abeau Chesnes, France) or prior 1U/ml of chondroitinase ABC treatment of the endothelial cells used for the assay.

mIECD³⁶ and mIE^{ICAM-1} were obtained by panning FCR3 IE preparations enriched by gelatin flotation on ScC2 and Sc3A4 Saimiri brain microvascular endothelial cells, which express either CD36 or ICAM-1, as described by Gay F. et al. (1995).

Placenta parasite populations that bound CSA on endothelial cells and placenta syncytiotrophoblasts were obtained by flushing 6 full-term placentas from Cameroonian women with malaria with a soluble 50 kDa CSA (Gysin J. et al. (1999)).

Induction of B Cell-Mediated Tolerance to CHO Cells and Normal Human Erythrocytes in Mice

B cells of 24-to 48-hours-old Balb/c mice (Iffa Credo, L'Arbresle, France) were rendered tolerant to normal human O⁻-erythrocytes (nE) or normal CHO-745 cells (nCHO) by antigenic overload.

The first sub-cutaneous injection into the dorsal region of 2×10⁹ nE or CHO-745 cells suspended in 0.2 ml of 0.9% NaCl was sufficient to induce B-cell mediated tolerance to these cells.

A booster intra-peritoneal injection of 5×10⁶ nE or 5×10⁵ CHO-745 cells suspended in 0.4 ml of 0.9% NaCl was performed 21 days after the initial injection.

Three weeks later, mice were tested for antibodies directed against surface antigens of nE or nCHO cells, by liquid-phase indirect immunofluorescence assay (1-IFA) with a 1:10 dilution of serum.

Immunization of Tolerant Mice with P. Falciparum-Infected Erythrocytes and CHO Cells Expressing DBL-3CSA

Mice with B-mediated cell tolerance for which no signal or only faint immunofluorescence (IF) observed with nE or nCHO cells, were selected for the specific immunization protocol. Approximately 5×10⁶ highly synchronized mIECSA or 5×10⁵ of transfected CHO cells expressing the DBL-γ3 domain of var_{CSA} were injected into each mouse. A second injection identical to the first was performed 3 weeks later.

Development of Mabs

Mice giving positive IFA results with mIE^{CSA} or CHO-DBL-γ3 were used for the development of mabs.

Mabs were produced by fusing mouse spleen cells with P3U1 cells as described elsewhere (Galfre G. et al. (1981), Kohler G. et al., (1975)).

IFA positive cells were cloned by limiting dilution, reassessed by 1-IFA and positive clones of interest were recloned by limiting dilution. Mabs that reacted strongly with the cell surface were expanded and isotyped by ELISA, using the ImmunoPure Monoclonal Antibody Isotyping Kit (Pierce, Rockford, Ill.61105 USA).

Indirect 1-IFA and ad-IFA

Two different types of indirect immunofluorescence assay were used for assessing the polyclonal antibody responses of mice and for the initial screening of monoclonal antibodies (mabs):

i) thin air dried infected blood smears (ad-IFA) and

ii) liquid-phase IFA (1-IFA) performed at +4° C. to prevent endocytosis with nE or nCHO cells and asynchronous and synchronized mIE^{CSA}, mIECD³⁶, mIE^{ICAM-1} and CHO-DBL-γ3/var_{CSA} transfectants.

Briefly: air-dried infected blood smears and fresh placenta cryosections were washed twice with PBS pH 7.4. Smears were incubated for 30 min at room temperature with 1 μg/ml DAPI (4, 6-diamidino-2-phenyl-indole dihydrochloride; Molecular Probes, USA) for nuclear staining and with mabs containing culture supernatants or 10 μg/ml purified mabs.

The smears were washed and incubated with a goat (Fab)′2 Alexa Fluor 488 labelled anti-mouse IgG or IgM (Molecular Probes, USA) at a dilution of 1/200 for an additional 30 min at room temperature. The slides were then washed and mounted in 30% (v/v) glycerol in PBS. For 1-IFA, we washed 10 μl of nE or asynchronous or synchronized mIE^{CSA}, mIE^{CD36}, mIE^{CAM-1} twice with culture medium without Albumax and incubated these cells in 5 μg/ml DAPI at +37° C. for 45 min.

The nE and IE were washed and incubated with culture supernatant or 10 μg/ml purified mab at +4° C. for 30 min., washed twice and incubated at +4° C. for an additional 30 min. with a goat (Fab)′2 Alexa Fluor 488 labeled anti-mouse IgG or IgM at a dilution of 1/200. In some cases, mIE^{CSA} were incubated with 100 μg/ml of trypsin or chymotrypsin before the addition of mabs, as previously described (Miller LH. et al. 1977).

For the staining of sequestrated mIE in placenta cryosections from women with malaria, we used the ad-IFA procedure with Evans blue counterstaining (1:10000 dilution) and simultaneous incubation with goat (Fab)′2 Alexa Fluor 488 labeled anti-mouse IgG or IgM (Molecular Probes, USA) at a dilution of 1/200.

Immunofluorescence staining was analyzed with a Nikon E800 microscope and images were acquired with a DDx Nikon camera.

ELISA

ELISA was performed with a slightly modified version of a published protocol (Perlmann H. et al., 1989).

Briefly, 96 well polystyrene microtiter plates (Nunc-Polylabo, Strasbourg, France) were coated with 10 μg/ml recombinant DBL-γ3var^{csa} (rDBL-γ3var^{csa}) produced in an insect cell expression system.

The plates were incubated overnight at 4° C., and unbound antigen was removed by washing with 0.05% Tween-20 in phosphatebuffered saline (PBST). Possible residual free sites were saturated by treatment with 1% BSA in PBS for 1 h at +37° C., and the plates were washed four times with PBST. We then added 100 μl of mab supernatant or 10 μg/ml purified mab to duplicate wells, and incubated the plates for 2 h at +37° C.

Wells were washed with PBST and the plates were incubated at +37° C. for 1 h with a peroxidase labellgoat anti-mouse IgG (Sigma, l'Isle Abeau Chesnes, France) diluted 1:4000 in PBST. Bound immunocomplexes were detected with o-phenylenediamine (Sigma, l'Isle Abeau Chesnes, France). Absorbance was read at 405 nm on a Multiskan Ascent ELISA reader (Labsystem, Helsinki, Finland). A positive result was considered to have been obtained for a mab (+) (Table 1) if the OD value was above the cutoff point set at 3 standard deviations (SD) above the mean background absorbance of P3U1 supernatant or unrelated mouse IgG isotypes or IgM.

Immunoprecipitation of ¹²⁵I surface labeled mIE^{CSA}

Mabs were used to immunoprecipitate the corresponding proteins from surface ¹²⁵I labeled synchronized IE^{CSA} trophozoite stage parasite extracts, as described by Buffet Paet al. (1999). IgM mab immune-complexes were recovered by incubation with an anti-mouse μ chain-specific goat IgG (Sigma, l'Isle Abeau Chesnes, France) followed by precipitation with protein G sepharose. A pool of sera from multiparous Cameroonian women (Gysin J, et al. (1999)) was used as a positive control and unrelated mouse IgM and IgG isotypes were used as negative controls.

Results

Induction of B Cell-Mediated Tolerance to Human Erythrocytes and CHO Cells

The number of Balb/c mice found to be tolerant after two injections of human erythrocytes or CHO cells was variable.

About 10 to 40% of the mice injected (depending on the series) with nE did not develop antibodies. Another 20 to 40 % gave faint IF and the other mice presented positive IF signals of various intensities.

The proportion of mice displaying B cell mediated tolerance to nCHO cells was much lower at 2 to 5%.

The best results for the production of specific antibodies against new surface antigens were obtained with “IF-negative” animals but satisfactory results were also achieved with animals that gave faint IF signals.

Mabs against P. Falciparum-Infected Erythrocyte Surface Antigens

The scores for specific mabs directed against surface-exposed antigens on infected erythrocytes in general were high and similar for mice immunized against trophozoite-IECSA or CHO cells expressing DBL-γ3. Typically, 20 to 60% of the 460 wells screened per fusion reacted with the surface of IE but not with nE. The initial selection of positive wells was based on the screening by liquid-phase indirect immunofluorescence assay (1-IFA) of mature parasite stage infected erythrocytes of the CSA adhesive phenotype. The 43 mabs chosen for this study, obtained from mice immunized against DBL-γ3 and against IE^{CSA} of the trophozoite stage, gave positive IF signals only with mature IE^{CSA} (mIE^{CSA}), but not with other parasites that express the CD36 or ICAM-1 adhesive phenotypes.

This IF was completely abolished by treating mIE^{CSA} with trypsin and chymotrypsin (100 μg/ml for 30 min at +37° C.). All 43 mabs reacted with the parasitophorous vacuole and vesicle like-structures (Maurer's clefts) of mIE^{CSA}.

Unlike 1-IFA, cross-reactivity with other adhesive phenotypes was observed for some mabs with air-dried parasites (see Table 1).

33% of the anti-mIE^{CSA} mabs cross-reacted with similar cell structures in mIE^{CD36} and mIE^{ICAM-1}. Anti-nE mabs were observed only at very low frequency (0.5%), demonstrating the efficacy of this novel immunization protocol. The mabs used were isotyped and it was found that the anti-mIE^{CSA} and anti-DBL-γ3 mabs were predominantly of the IgM isotype: 75% of anti-CHO-DBL-γ3CSA mabs were IgM, and 25% were IgG2a. For anti-mIE^{CSA} mabs, 66.7% were IgM, 25% were IgG2a and 8.3% were IgG1 (see Table 1). All mabs carried a κ-light chain.

TABLE 1
Characterization of anti-mIEcsa and anti-DBL-γ3 mabs by IFA and ELISA. The
11 clones shown correspond to 25% of the positive clones obtained after fusion.
(+) positive (−) negative.

| | | 1-IFA | | | ad-IFA | | | ELISA |
|---|---|---|---|---|---|---|---|---|
| mAbs | Isotype | CSA | CD36 | ICAM-1 | CSA | CD36 | ICAM-1 | rDBL-γ3 |
| anti-mIECSA | | | | | | | | |
| 3F3/C2/C1 | IgG2a | + | − | − | + | − | − | + |
| 1H6/A4 | IgG1 | + | − | − | + | − | − | + |
| 2H5/F10 | IgM | + | − | − | + | − | − | − |
| 1E5/D6 | IgM | + | − | − | + | − | − | + |
| 1E5/EE4 | IgM | + | − | − | + | + | + | + |
| 2A11/E4 | IgM | + | − | − | + | + | + | + |
| anti-CHO-DBL-γ3 | | | | | | | | |
| 1C5/D12 | IgG2a | + | − | − | + | − | − | + |
| 1B11/A5 | IgM | + | − | − | + | − | − | + |
| 1B4/D4 | IgM | + | − | − | + | − | − | − |
| 2F5/G10 | IgM | + | − | − | + | − | − | + |
| 4F10/C8 | IgM | + | − | − | + | − | − | − |

The reactivity of mabs with parasite surface molecules was investigated using extracts of synchronized I¹²⁵ surface labeled mIE^{CSA}. Both types of mab, anti-mIE^{CSA} and anti-DBL-γ3, immunoprecipitated a molecule of approximately 400 kDa, previously shown to correspond to PfEMP1CSA (Buffet PA. et al.1999).

DBL-γ3CSA is the Target of Most Anti-mIECSA Mabs

The specificity of anti-mIE^{CSA} mabs for PfEMP1^{CSA} was further analyzed by testing their reactivity to the domain that binds to CSA. To this end, a recombinant rDBL-γ3/var^{CSA} was produced by an insect-cell expression system.

This recombinant consisted of the DBL-γ3 region expressed by the CHO transfectant that specifically binds CSA (Buffet Pa. et al.1999). The recombinant rDBL-γ3/var^{CSA} protein inhibits the cytoadhesion of mIE^{CSA} to endothelial cells and syncytiotrophoblasts by more than 60%. The rDBL-γ3 var^{CSA} protein reacted specifically with 15 of 23 anti-mIE^{CSA} mabs in ELISA. As expected, almost all anti-CHO-DBL-γ3 mabs recognized rDBL-γ3/var^{CSA} (85%). The intensity of surface IF and the absorbance values obtained in ELISA were not correlated (Table 1). We conclude that the DBL-γ3 domain not only mediates adhesion to CSA but also acts as an immunodominant region of PfEMP1-CSA.

Pan-Reactivity of Anti-CHO-DBL-γ3CSA and Anti-mIE^{CSA} Mabs

Two mabs, 2H5/D3 and 1B11/A5, respectively anti-mIE^{CSA} and anti-CHO-DBL-γ3, were arbitrarily chosen to investigate the reactivity with multiple variants of a number of CSA-binding parasites from different geographic regions (Brazil, Thailand and West Africa). Surface staining by 1-IFA showed that all 7 laboratory strains analyzed (Table 2) reacted with both mabs, 2H5/D3 and 1B11/A5, at varying degrees (2% to 98%) in laboratory strains not previously selected for CSA-binding (Table 2).

TABLE 2
Analysis of pan-reactivity of mabs 2H5/D3 and 1B11/A5 and
cytoadherence phenotypes of strains from different endemic areas.

| | 2H5/D3 | | 1B11/A5 | | | |
|---|---|---|---|---|---|---|
| | Before | After | Before | After | CSA | Case ABC |
| Strains | panning | panning | panning | panning | (100 μg/ml) | (1 U/ml) |
| B358* | 2 | >94 | 2 | >94 | 96 | 90 |
| BXII* | 5 | >94 | 5 | >94 | 95 | 92 |
| FCBR* | 5 | >94 | 4 | >94 | 95 | 96 |
| SUK** | 98 | >94 | 95 | >94 | 90 | 91 |
| H** | 2 | >94 | 3 | >94 | 92 | 93 |
| IBR** | 97 | >94 | 95 | >94 | 90 | 95 |
| FCR3* | 0, 3 | >94 | 34 | >94 | 91 | 96 |

Panning of each of these parasite strains on Sc17 cells, which carry CSA as the only adhesion receptor, resulted in a considerable enrichment in mIE which reacted with both mabs (>94%) in all CSA binding strains. Cytoadhesion inhibition assays on Sc1D cells with these 6 panned parasite subpopulations resulted in the inhibition of mIE adhesion, by 90% to 96%, by 100 μ/ml CSA or 1U/ml of chondroitinase ABC treatment of the endothelial cells (Table 2). The reactivity of 2H5/D3 and 1B11/A5 with placental isolates from 6 different malaria infected women was investigated using placental tissue cryosections. All sections showed large numbers of adhering parasites and gave strong signals with the two mabs. The reactivity was completely inhibited in the presence of soluble CSA and chondroitinase ABC treatment.

However, only a fraction of the pigmented erythrocytes in the placenta were stained with 2H5/D3 and 1B11/A5 (approximately between 40 to 60%), suggesting the presence of parasites that might bind to a distinct placental receptor such as the Fc/IgG receptor or hyaloronic acid. We conclude that the two mabs, 2H5/D3 and 1B11/A5, directed against FCR3 DBL-γ3^{CSA}, define cross-reacting epitopes that are conserved in geographically and genetically distinct CSA-binding parasite populations, including clinical isolates, and are involed in human placental infection.

### Example 2

Material and Methods

Induction of Anti-Human O⁺nRBCs Immuno-Tolerance

24 to 48 hours old Balb/c (IFFA-CREDO, France) are inoculated subcutaneously with 1.5 to 2×10⁹ normal human O⁺ red blood cell (NRBC) pellet previously washed 6× in 0.9% NaCl (O⁺nRBCs) in the back region. To avoid a possible reflux of the injected cells, the same quantity of nRBCs can also be divided into two identical parts and be injected at a 24 hours interval subcutaneously in the back region.

21 days after the first antigen injection the animal are boosted by an intra peritoneal injection of 5×10⁶ O⁺nRBCs. 21 days later the animal are screened for anti-O⁺nRBCs antibodies by using a liquid phase IFA at 4 C. 10μl of O⁺nRBCs are resuspended during 30 min in a ¼ diluted mouse plasma (decomplemented at 56 C for 30 min) at 4 C. The O⁺nRBCs pellet is washed 3× with 500 ,μl of cold RPMI 1640 (Sigma, France). The pellet is resuspended in 100 μl Alexa Fluor 488 labeled goat Fab′2 anti-mouse IgG (Molecular-Probes, Eugene, Orego, ref A-11017) at a dilution of 1:300 in RPMI. The Fab′2 anti-mouse IgG was preabsorbed 3× with O⁺nRBCs (about 3×40 μl/1ml). A Nikon E800 microscope with an epifluorescence objective 100×Oil can be used.

Immunization of Mice B Cell Tolerant to Human Erythrocytes (or CHO Cells)

IFA negative animals were considered to be “tolerant” (no detectable antibody response) against O⁺nRBCs.

These animals were then injected intraperitoneal with 5×10⁶ Plasmodium falciparum infected human O⁺nRBCs (PRBC) which have been previously selected by panning for binding to the adhesion receptor chondroitin sulfate A (CSA).

Either young ring stage infected erythrocytes (expressing parasite surface molecules RSP 1 and RSP2) or pigmented mature stage infected erythrocytes (expressing parasite surface molecules PfEMP1, Rifin and Clag) were prepared and injected into the animals.

21 days later the mice are boosted with same quantity and the corresponding PRBCs by the same route.

14 days later the animal were assayed for antibodies directed against surface exposed antigens of PRBCs.

15 μl asynchronous FCR3-PRBC of the CSA-, or the CD36- and ICAM-1-phenotype (Robert et al., 1995) obtained from continuous culture are incubated for 1 hour at +37 C with 50 μl dapi at 40 μg/ml (Molecular Probe).

After centrifugation the pellet is resuspended in 50 μl of a ¼ dilution of plasma and incubated 30 min at 4 C.

After 3 washing steps with 500 μl of cold RPMI the pellet is resuspended and incubated with 50 μl Alexa Fluor 488 goat Fab′2 anti-mouse IgG (Molecular-Probes) at a dilution of 1/300 for 30 min at 4 C.

After washing 3× with 500 μl of cold RPMI, 15 μl of a suspension of PRBCs in RPMI are mounted between a slide and coverslip.

The IF lecture was done with a Nikon E800 microscope with an epifluorescence objective 100×Oil.

Mice which had developed antibodies against antigens expressed on the surface of ring-stage-PRBC or anti-mature forms infected erythrocytes of the CSA phenotype were then boosted a second time as previously (3th PRBC injection).

Two days later the animals were sacrificed and exanguinated and the spleen recovered.

Fusion at the ratio of 1 spleen cell/3 P3U1 myeloma cells was done by following elsewhere described procedure (Kohler Milstein 1975; Hales 1977; Galfre and Milstein 1981; Gysin et al. 1985) and the cell suspension plated in five plates of 96 flat bottom wells (Coming) (Kohler Milstein 1975; Hales 1977; Galfre and Milstein 1981; Gysin et al. 1985).

Two hybridomas cell lines were selected and their characteristics of monoclonal antibodies C10 and B4 produced by these two hybridoma cell lines are: both react with the native P. falciparum proteins at the surface of ring-infected erythrocytes but not with mature trophozoite and schizont-infected erythrocytes.

Both inhibit the adhesion of ring-infected erythrocytes. B4 inhibits also the re-invasion of merozoites of erythrocytes.

Two cell lines have been deposited on Feb. 23, 2001.

The hybridoma secreting antibody B4, Pf26G1/B4, was deposited at Collection Nationale de Cultures de Microorganismes (CNCM) on Feb. 23, 2001, under accession number I-2635.

The hybridoma secreting antibody C10, Pf 26G1/C10, was deposited at Collection Nationale de Cultures de Microorganismes (CNCM) on Feb. 23, 2001, under accession number I-2636.

Wells with growing clones were screened by above mentioned liquid phase IFA. Positive wells were cloned by limited dilution in the presence of Hybridoma cloning Factor (IGEN, Tebu, France ) and screened by surface IFA.

Positive clones were expanded and the IgG isotyped. mabs of the isotypes IgG1, IgG2a and IgG3 were obtained. Clones are expanded either by culture or by injecting pristine treated mice. The average frequency of positive motherwells (not cloned) oscillated between 10 to 30 positive wells (recognizing conformational antigens located at the surface of RBC) for 50 screened wells. Some supernatants contain antibodies that are directed against O⁺nRBCs. In contrary, by using chemical technique or the technique described by Smith et al. (1995), the positive cells are about 1/200 to 1/500 instead of 10 to 30/50 as obtained in the method described herein.

TABLE 3
Immunization results
of Balb/c mice B cell tolerant to O⁺nRBCs or CHO cells.

| | | Phenotype | Stage | Cytoadhesion |
|---|---|---|---|---|
| Antiserum/Mabs | Surface IF | Specific | Specific | Inhibition |
| anti-troph from | yes | yes | yes | Nd |
| PRBC/CSA | | | | |
| anti-ring-stage | yes | No | yes | yes |
| PRBCs/CSA | | | | |
| anti-CHO-DBL- | yes | yes | yes | Nd |
| 3/var^{CSA} | | | | |

nd = not done

### Example 3

### Anti-HBV Antibodies

The immunization technique was carried out with CHO clone 37 BA5 CNCM I-1772 cells, which produce the small and medium-size proteins of the hepatitis B virus envelope. The proteins are assembled and secreted in the form of 22 nm particles bearing the HBs and preS2 antigens. After immunization and fusion (using the technique described in example 1), the presence of antibodies (from the second week after fusion) was demonstrated by means of ELISA with the recombinant HBs antigen purified from CHO cells, which was identical to the HBs antigen purified from human plasma of subtype ay. Anti-HBs antibodies were detected in 17.7% of the original wells. From 27.6% of the original wells tested positive with the ay and ayw antigens, 51.7% were specific for ay and 20.7% for ayw.

### Example 4

### Cancer Treatment

The production of antibodies specifically directed against tumour neo-antigens, by first rendering mice tolerant to “normal” host cells of the same type. The antibodies obtained could be use for the detection in vitro of a neo-antigen, by FacSCAN in free cells, and by immunofluorescence (IF) in biopsy samples.

More advantageously, a radioisotope may be attached to such antibodies, which may be used for tumour diagnosis and the detection of disseminated metastases, in an ad hoc manner. Similarly, the binding of certain toxic molecules to antibodies of this type could be used to destroy cancer cells with a minimum of collateral effects.

Obviously, numerous modifications and variations of the present invention are possible in light of the above teachings. It is therefore to be understood that within the scope of the appended claims, the invention may be practiced otherwise than as specifically described herein.

### REFERENCES

Buffet Pa., Gamain B, Scheidig C, et al. Plasmodium falciparum domain mediating adhesion to chondroitin sulfate a: a receptor for human placental infection. Proc Natl Acad Sci U S A.1999;96:12743-12748.

Emery et al., Antibody enginering, 2^{nd} edition, Carl A. K. Borrebaeck, Oxford, 1995

Felici et al., J. Mol. Biol., 1991, Vol. 222, p. 301.

Fusai T, Parzy D, Spillmann D, et al. Characterisation of the chondroitin sulphate of Saimiri brain microvascular endothelial cells involved in Plasmodium falciparum cytoadhesion. Mol Biochem Parasitol. 2000;108:25-37.

Galfre G., and Milstein C. 1981. In: Methods in Enzymology, vol. 73, eds. J. J. Langone and H. Van Vunakis (Academic Press, New York) p. 3.

Gay F, Robert C, Pouvelle B, Peyrol S, Scherf A, Gysin J. Isolation and characterization of brain microvascular endothelial cells from Saimiri monkeys. An in vitro model for sequestration of Plasmodium falciparum infected erythrocytes. J Immunol Methods. 1995;184:15-28.

Gysin J., Roussilhon C., et Pauillac S. 1985. Measurement of squirrel monkey IgG levels by a two-site sandwitch radioimmunoassay with monoclonal antibodies. J. Immunol. Methods. 82: 295-301.

Gysin J, Pouvelle B, Fievet N, Scherf A, Lepolard C. Ex vivo desequestration of Plasmodium falciparum-infected erythrocytes from 21 human placenta by chondroitin sulfate A. Infect Immun. 1999;67:6596-6602.

Hales A. 1977, Somatic Cell Genet. 3, 227.

Heidrich H G, Mrema J E, Vander Jagt D L, Reyes P, Rieckmann K H. Isolation of intracellular parasites (Plasmodium falciparum) from culture using free-flow electrophoresis: separation of the free parasites according to stages. J Parasitol. 1982;68:443-450.

Kohler G., and Milstein C. 1975. Nature (London) 256-495.

Lambros C, Vanderberg J P. Synchronization of Plasmodium falciparum erythrocytic stages in culture. J Parasitol. 1979;65:418-420.

Miller L H, Haynes J D, McAuliffe F M, Shiroishi T, Durocher J R, McGinniss M H. Evidence for differences in erythrocyte surface receptors for the malarial parasites, Plasmodium falciparum and Plasmodium knowlesi. J Exp Med. 1977;146:277-281.

Parzy D, Fusai T, Pouvelle B, et al. Recombinant human thrombomodulin(csa+): a tool for analyzing Plasmodium falciparum adhesion to chondroitin-4-sulfate. Microbes Infect. 2000;2:779-788.22.

Perlmann H, Perlmann P, Berzins K, et al. Dissection of the human antibody response to the malaria antigen Pf155/RESA into epitope specific components. Immunol Rev. 1989;1 12:115-132. Review.

Pouvelle B, Fusai T, Lepolard C, Gysin J. Biological and biochemical characteristics of cytoadhesion of Plasmodium falciparum-infected erythrocytes to chondroitin-4-sulfate. Infect Immun. 1998;66:4950-4956.

Ren E. C., Ann. Acad. Med. Singapore, 1991, 20, 66-70.

Robert C, Pouvelle B, Meyer P, et al. Chondroitin-4-sulphate (proteoglycan), a receptor for Plasmodium falciparum-infected erythrocyte adherence on brain microvascular endothelial cells. Immunol. 1995;146:383-393.

Smith, J. D., Chitnis, C. E., Craig, A. G., Roberts, D. J., Hudson-Taylor, D. E., Peterson, D. S., Pinches, R., Newbold, C. I., and Miller, L. H. (1995). Switches in expression of Plasmodium falciparum var genes correlate with changes in antigenic and cytoadherent phenotypes of infected erythrocytes, Cell 82, 101-10.

All of the publications cited above are incorporated herein by reference.

1. A process for preparing monoclonal antibodies, comprising:
rendering an animal tolerant to an eukaryotic cell in a first state;
detecting said tolerant animal;
immunizing said tolerant animal, by injection of the eukaryotic cell in a second state carrying a neo-antigen or a non-self antigen;
fusing B cells of said immunized mice with a myeloma cell line; and
selecting the hybridoma expressing antibodies against said neo-antigen or non-self antigen.

2. The process of claim 1, further comprising:
(f) optionally culturing the selected hybridoma and purifying the monoclonal antibodies.

3. The process of claim 1, wherein said neo-antigen or non-self antigen is selected from the group consisting bacterial, fungi, parasitic, and cancer antigens and any antigen and used by the normal or pathological development of the cell.

4. The process of claim 1, wherein the antibodies are further humanized.

5. A monoclonal antibody susceptible to be prepared by the process of claim 1.

6. The monoclonal antibody of claim 5, wherein said antibody is further humanized.

7. A native or conformational antigen capable of reacting with a monoclonal antibody produced by the process according to claim 1.

8. A process for screening an active molecule capable of reacting specifically with a monoclonal antibody according to claim 5.

9. A process for selecting a native or conformational antigen, comprising:
(a) rendering an animal tolerant to an eukaryotic cell in a first state;
(b) detecting said tolerant animal;
(c) immunizing said tolerant animal, by injecting said eukaryotic cell in a second state carrying a neo-antigen or a non-self antigen;
(d) preparing an hybridoma against said neo-antigen or non-self antigen;
(e) selecting the hybridoma expressing antibodies against said neo-antigen or non-self antigen;
(f) contacting the monoclonal antibody produced by the hybridoma of (e) with an antigenic preparation; and
(g) selecting the complex formed between said monoclonal antibody and the conformational native antigen of interests.

10. The process of claim 9, further comprising:
revealing the complex; and
optionally, separating the antibody from the conformational antigen from the complex.

11. The process of claim 9, wherein said neo-antigen or non-self antigen is selected from the group consisting of bacteria, fungi, parasitic, fungal, cancer antigens and any antigen induced by the normal or pathological development of the cell.

12. An active molecule according to claim 7, which is a component for a diagnostic detection of the presence or absence of antibodies in a serum of an animal, including human.

13. An active molecule according to claim 7, which can compete with the neo or non-self antigen of the virus, the bacteria, the fungi, the parasite or the cancer present at the surface of cells or induced by the normal or pathological development of the cell.

14. An active molecule according to claim 7, capable of inducing an immune response in vivo or in vitro against a bacterial or viral or parasite infection, against a cancer or any pathological development of the cell inducing neo-antigen development.

15. Use of the antibody according to claim 5 in the preparation of a composition for the immunization or the treatment of a human or an animal for a virus, bacteria, fungi or parasite infection or cancer.

16. Use of the antibody according to claim 5 in the preparation of a composition for diagnosing a viral, bacterial, parasite, fungal, infection, a cancer or any development of the cell inducing neo-antigen development.

17. A process for targeting eukaryotic cells carrying a neo-antigen or a non-self antigen wherein said process uses monoclonal antibodies directed against said neo-antigen or non-self antigen obtained by the process of claim 1.

18. The process of claim 16 or 17, wherein said monoclonal antibodies are further labeled.

19. The process of claim 17, wherein said monoclonal antibodies are further coupled to a molecule toxic for the targeted cells.

20. The hybridoma Pf 26G1/B4 deposited at Collection Nationale de Cultures de Microorganismes (CNCM) on Feb. 23, 2001, under accession number 1-2635.

21. The hybridoma Pf 26G1/C10 deposited at Collection Nationale de Cultures de Microorganismes (CNCM) on Feb. 23, 2001, under accession number I-2636.

22. A process for screening active molecule capable of reacting specifically with the conformational, native, poorly immunogenic, minor antigen obtained by the process of claim 9.

23. A process according to claim 1, wherein the frequency of obtained hybridoma cell lines having the property of recognizing selectively a conformational antigen or a native antigen is up to 200 times greater than hybridoma cell lines obtained by classical techniques.

24. A hybridoma which secretes an antibody having the same epitope specificity as the antibody produced by hybridoma Pf 26G1/B4 deposited at Collection Nationale de Cultures de Microorganismes (CNCM) on Feb. 23, 2001, under accession number I-2635.

25. A hybridoma which secretes an antibody having the same epitope specificity as the antibody produced by hybridoma Pf 26G1/C10 deposited at Collection Nationale de Cultures de Microorganismes (CNCM) on Feb. 23, 2001, under accession number I-2636.

26. A hybridoma susceptible to be obtain with step (e) of the process according to claim 1 or to claim 9.

27. A process according to claim 1 or to claim 2, in which the animal is a mouse.

28. A conformational antigen selected and characterized by its capacity to react with monoclonal antibody obtained by a process which is 200 uptimes greater successful than classical process to obtain similar hybridoma.

29. Kit for the detection of antigens, comprising at least a monoclonal antibody obtained by the process of claim 1.
